(19) United States
(12) Patent Application Publication
(10) Pub. No.: US 2021/0282904 A1
(43) Pub. Date: Sep. 16, 2021

(54) One single drill dental implant system and Method for Use Thereof

(71) Applicant: Chun Leon CHEN, Taipei City (TW)

(72) Inventors: Chun Leon CHEN, Taipei City (TW); Le Chen Chen, Las Vegas, NV (US); Sho Chin Chen, Las Vegas, NV (US); Hong Yui Chen, Las Vegas, NV (US); Yang How Chen, Las Vegas, NV (US); Tseu Min Chen, Las Vegas, NV (US)

(21) Appl. No.: 16/865,421

(22) Filed: May 4, 2020

(30) Foreign Application Priority Data
Mar. 16, 2020 (CN) 202010180230.6

(51) Int. Cl. A61C 8/00

(52) U.S. Cl. CPC A61C 8/0089; A61B 17/1673; A61C 8/0025; A61C 8/0024

(57) ABSTRACT

The present invention provides one single drill dental implant system and method for use thereof. The system comprises a multistep dental implant drill and a side cutting capability dental implant, and the multistep dental implant drill engaged with a dental handpiece to perform a dental implant step. Further, the method comprises a positioning step of aligning a tip of a drill bit portion of a multistep dental implant drill with a preset dental implant position; a drilling step of rotating the multistep dental implant drill by a dental handpiece to drill an implant hole; an expanding step of drilling the alveolar bone through a plurality of steps and cutting members of the drill bit portion, so that the diameter of the implant hole is gradually enlarged to be smaller than the diameter of a dental implant; and an implantation step of implanting the dental implant into the implant hole.

### (a) Technical Field of the Invention

The present invention relates to one single drill dental implant system and method for use thereof, and more particularly to one single drill dental implant system for immediate implant placement and method for use thereof.

### (b) Description of the Prior Art

A dental implant is made of titanium or zirconium dioxide, it is implanted in the patient's oral alveolar bone, and its function similar to the roots of the original teeth. After the dental implant is tightly integrated with the bone, artificial crown is installed on the roots made of titanium or zirconium dioxide to restore esthetic appearance of tooth and chewing function. However, if the jaw bone is too thin, a dentist can use bone graft materials to perform a guided bone regeneration or a sinus lifting. Therefore, it takes a long time to wait until the dental implant is osseointegrated with the bone before making the artificial crown. Secondly, the complete dental implant structure is composed of an implant body, a connector (abutment) and the artificial crown, and the three parts can be fixed by screws or connectors to replace the patient's original teeth.

Generally, when dental implants are installed, it is necessary to perform an osteotomy to form a circular hole with an appropriate sized in the alveolar bone for implanting a dental implant. Therefore, during dental implant operation, the surgical tools affect the success or failure of the dental implant operation and the stability of the dental implant after the operation. Generally, dental implant operation can comprise the steps of positioning, drilling, expanding, tapping, and implanting. When the dentist places the dental implant, it needs to be positioned on the alveolar bone before drilling and cutting to drill holes that match the size of the dental implant. Finally, the dental implant is installed into the alveolar bone.

Traditionally, an implant hole formed by an osteotomy is drilled by different multiple drill bits, and the implant hole is drilled by a countersink drill until the diameter is equal to that of a threaded dental implant. The traditional method of drilling holes requires multiple changes of different sizes drill bits, which increases the operation time, chances of infection, risk of excessive bleeding, patients discomfort, and post-operation complications. Secondly, during the drilling and cutting steps, great strain energy, friction energy, and thermal energy will be generated, which will cause damage to material of the object to be cut. According to research by scholars, the heat generated by the cutting process may be compromised success of osseointegration after inserting dental implants.

Furthermore, if the size of the implant hole is too large, the dental implant may be mobilized during implantation. In order to stabilize the dental implant, fillers need to be added to the implant hole, which increases the operation time of dental implants into the alveolar bone. On the contrary, if the size of the implant hole is too small, a large amount of bone debris will be generated when the dental implant is implanted into the drill hole, which makes it difficult for the dental implant to be inserted correctly in the implant hole, and increase the osseointegration healing time, or possible fail the entire osseointegration of the dental implant.

### SUMMARY OF THE INVENTION

In view of the shortcomings of the above-mentioned dental implantation technology, the main purpose of the present invention is to provide one single drill dental implant system and method for use thereof, which can directly insert dental implants into the alveolar bone, thereby achieving the effect of shortening the operation time, decrease infection rate, and decrease patient discomfort. Secondly, during the dental implantation process, excess bone debris can be effectively discharged, so that the alveolar bone and the dental implant can be tightly connected to ensure that the dental implant does not mobilize, thereby improving the success rate of operation and osseointegration to achieve better initial stability.

To achieve the above object, the present invention provides one single drill dental implant system and method for use thereof, and the one single drill dental implant system comprises a multistep dental implant drill and a dental implant. First, the multistep dental implant drill is engaged with a dental handpiece, and the multistep dental implant drill comprises a rod portion, a stopper, and a drill bit portion. Further, the stopper is connected to the rod portion and the drill bit portion, and the drill bit portion comprises a plurality of steps with a diameter decreasing from top to bottom, at least one cutting flute and at least one tip. The cutting flute penetrates the outer surface of the drill bit portion and extends from the bottom of the drill bit portion to the bottom of the stopper. Secondly, the dental implant comprises a implant fixture body, a tooth root neck and a plurality of side-cutting grooves. The implant fixture body has a fine thread portion, a coarse thread portion, and the plurality of side-cutting grooves. The fine thread portion is located on the upper surface of the implant fixture body, and the coarse thread portion is located on the lower surface of the implant fixture body. Further, the side-cutting grooves are distributed on the coarse thread portion and penetrate the outer surface of the implant fixture body, and the side-cutting grooves extend from the bottom of the implant fixture body to the bottom of the fine thread portion. In addition, the tooth root neck has a joint hole, and the tooth root neck is located on the top of the implant fixture body; wherein the tip of the multistep dental implant drill is aligned with a preset implant hole on an alveolar bone to drill an implant hole and insert the dental implant. Furthermore, the length of the fine thread portion is in a range of 5% to 15% based on the total length of the implant fixture body; and the length of the coarse thread portion is in a range of 85% to 95% based on the total length of the implant fixture body.

In some embodiments, the multistep dental implant drill has a point angle which is set at the top of the drill bit portion, and point angle is in a range of 70 degrees to 120 degrees. The point angle is an important design parameter, and its size is defined by top angle of the drill bit portion. The design of the point angle will determine whether the multistep dental implant drill can perform stable cutting to form the implant hole, and has a significant impact on the counter force and cutting energy during cutting. In a preferred embodiment of the present invention, the point angle is 70 degrees to 80 degrees, and in another preferred embodiment, the point angle is 90 degrees to 118 degrees.

In some embodiments, the multistep dental implant drill includes two of the cutting flutes to be respectively a first cutting flute and a second cutting flute, and the first cutting flute and the second cutting flute penetrate outer surface of drill bit portion; the first and second cutting flutes extend from the bottom of the drill bit portion to the bottom of the stopper; and the second cutting flute is opposite to the first cutting flute.

In some embodiments, each of the plurality of steps and two sides of the first cutting flute and the second cutting flute form a plurality of blades.

In some embodiments, the blades have a plurality of cutting members.

In some embodiments, the stopper has a stopping line, and the stopping line is arranged around the bottom of the stopper. A dentist can judge the depth of the multistep dental implant drill into the alveolar bone through a stopping line. When the multistep dental implant drill is drilled into the alveolar bone until the stopping line contacts the alveolar bone, the implant hole is completed, so as to avoid drilling implant hole too deep and hurting the patient's vital tissue like nerves and arteries or drilling implant hole too shallow so that the dental implants cannot be implanted. In addition, when the dentist uses a dental surgical guide tubing for drilling, the stopper of the multistep dental implant drill has a certain thickness; therefore, when the dentist drills, the multistep dental implant drill is drilled into the alveolar bone until the stopper is caught on the ring of the dental surgical guide tubing, the drilling is completed.

To achieve the other above object, the present invention provides a method for using one single drill dental implant system comprising the steps of a positioning step, a drilling step, an expanding step and an implantation step. First, the positioning step is that a tip of a drill bit portion of a multistep dental implant drill is aligning with a preset dental implant position. Secondly, the drilling step is that the multistep dental implant drill is rotated by a dental handpiece to drill an implant hole in the alveolar bone. Further, the expanding step is that the drill bit portion is drilled into the alveolar bone through a plurality of steps and a plurality of cutting members of the drill bit portion, so that the diameter of the implant hole is gradually enlarged to be smaller than the diameter of a dental implant. Furthermore, the implantation step is that the dental implant is implanted into the implant hole.

In some embodiments, the method further comprises a further drilling step is that a stopping line of a stopper is used to determine the depth of the drill bit portion into the alveolar bone after the expanding step; and the drilling is completed, when the stopping line contacts the alveolar bone.

In some embodiments, the alveolar bone is cut by a cutting surface of the side-cutting groove of the dental implant when the dental implant is implanted, and the side-cutting groove is filled with patient's own bone debris and locks the dental implant, any excess patient's own bone debris can be washed out. In addition, the bone pressure can be relieved by the side-cutting groove to prevent bone necrosis.

In one single drill dental implant system, when the drill bit portion with multistep drills into the alveolar bone, it will form a step formation on the bone. The dental implant with the sharp side-cutting grooves will strongly locks the implant fixture body with all the different levels of steps. Thus give the tight initial stability. Therefore, one single drill dental implant system of the present invention can be used in the immediate extraction socket, immediate implantation and immediate temporary provisional.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic view of a multistep dental implant drill according to an embodiment of the present invention;

FIG. 2 is a schematic view of a multistep dental implant drill according to another embodiment of the present invention;

FIG. 3 is a schematic view of a dental implant according to Example 1 of the present invention;

FIG. 4 is a schematic view of a dental implant according to Example 2 of the present invention;

FIG. 5 is a schematic view of a dental implant according to Example 3 of the present invention;

FIG. 6 is a schematic view of a dental implant according to Example 4 of the present invention;

FIG. 7A is a top view of the dental implant according to Example 1 of the present invention;

FIG. 7B is an another stereoscopical schematic view of the dental implant according to Example 1 of the present invention;

FIG. 8 is a schematic view of multistep dental implant drill drilled into alveolar bone according to the present invention;

FIG. 9 is a schematic view of the dental implant implanted into an implant hole in the alveolar bone according to Example 1 of the present invention;

FIG. 10 is a schematic view of the dental implant implanted into an immediate extracted tooth socket in the alveolar bone according to Example 1 of the present invention;

FIG. 11 is a schematic view of dental implant implanted into a healed alveolar bone according to the present invention;

FIG. 12 is a flow chart of a method for using one single drill dental implant system of the present invention;

FIG. 13 is a schematic view of a set of dental implant drill required for drilling holes in current osteotomy; and

FIG. 14 is a schematic view of multistep dental implant drill of the present invention formed by five different dental implant drills.

### DETAILED DESCRIPTION OF THE PREFERRED EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

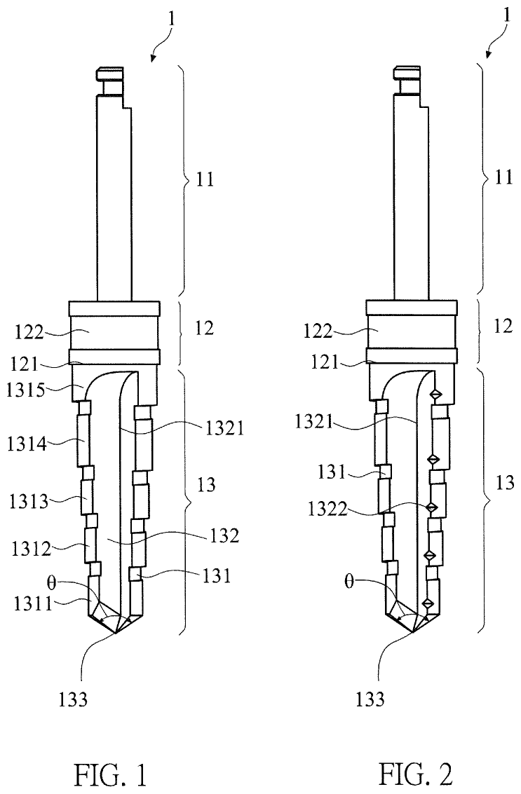

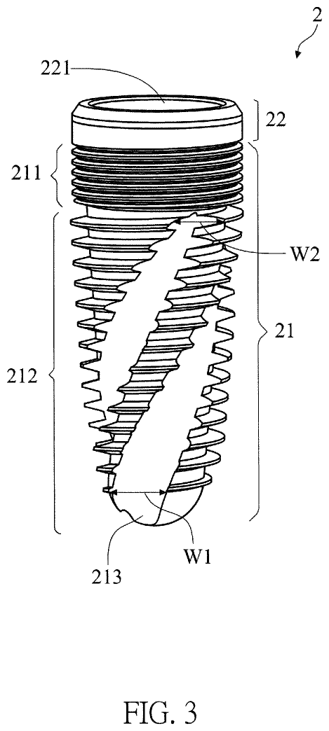

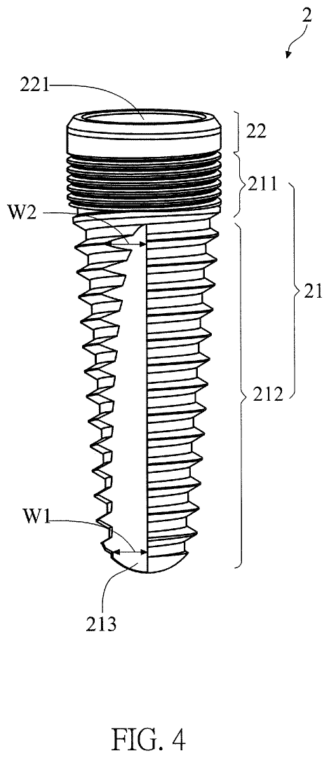

Please refer to FIG. 1 to FIG. 4, FIG. 1 is a schematic view of a multistep dental implant chill according to an embodiment of the present invention; FIG. 2 is a schematic view of a multistep dental implant drill according to another embodiment of the present invention; FIG. 3 is a schematic view of a dental implant according to Example 1 of the present invention; and FIG. 4 is a schematic view of a dental implant according to Example 2 of the present invention.

As shown in FIG. 1 to FIG. 4, the present invention provides one single drill dental implant system which comprises a multistep dental implant drill 1 and a dental implant 2. First, the multistep dental implant drill 1 is engaged with a dental handpiece, and the multistep dental implant drill 1 comprises a rod portion 11, a stopper 12, and a drill bit portion 13. Further, the stopper 12 is connected to the rod portion 11 and the drill bit portion 13, and the stopper 12 does not have cutting flute for safeguard, and the drill bit portion 13 comprises a plurality of steps 131 with a diameter decreasing from top to bottom, at least one cutting flute 132 and at least one tip 133. The drill bit portion 13 comprises an initial drill 1311, a second drill 1312, a third drill 1313, a fourth drill 1314 and a counter sink 1315; wherein the initial drill is used for marking the position of the dental implant 2, and the plurality of steps 131 with a diameter decreasing from the counter sink 131 to the second drill 1312. Further, the initial drill 1311 has a smallest diameter and a shortest cutting surface length; and the counter sink 1315 has a biggest diameter and a longest cutting surface length. The cutting flute 132 has side-cutting capability for grinding the dental bone. Since the multistep dental implant drill 1 has the plurality of the steps 131, only one single drill can be used to complete the drilling, so the cost of the dental implant drill can be reduced. In addition, the side of the steps131 of the multistep dental implant drill 1 has a side-cutting function; therefore, when the drilling angle is skewed, the angle can be slowly corrected by the side of the steps131. Further, the cutting flute 132 penetrates the outer surface of the drill bit portion 13 and extends from the bottom of the drill bit portion 13 to the bottom of the stopper 12. In a preferred embodiment of the present invention, the multistep dental implant drill 1 comprises two cutting flutes 132, and the two cutting flutes 132 are a first cutting flute and a second cutting flute. The first cutting flute and the second cutting flute penetrate the outer surface of the drill bit portion 13, and extend from the bottom of the drill bit portion 13 to the bottom of the stopper 12. Furthermore, the second cutting flute is disposed on the opposite side of the first cutting flute, that is, the second cutting flute is disposed opposite to the first cutting flute. In a preferred embodiment of the present invention, the multistep dental implant drill 1 comprises three cutting flutes 132, and the three cutting flutes 132 are the first cutting flute, the second cutting flute, and a third cutting flute, respectively. The cutting flute 132 can spray water to cool down when the temperature is too high, and cold water will flow into the dental implant drill from the cutting flute 132 to reduce the temperature. In addition, each of the steps 131 and the two sides of the first cutting flute and the second cutting flute are formed with a plurality of blade 1321, and the blade 1321 can fix the movement direction of the multistep dental implant drill 1 to avoid displacement of the multistep dental implant drill 1. Then, the at least one tip 133 has a symmetrical cut surface for fixing the drilling direction and preventing the multistep dental implant drill 1 from sliding. In a preferred embodiment of the present invention, the multistep dental implant drill 1 can include two tips 133, which can improve the stability of the multistep dental implant drill 1 during rotation to avoid the bounce of the multistep dental implant drill 1, and can accurately align the position of the implant hole to facilitate subsequent implantation steps.

Furthermore, the difference between FIG. 1 and FIG. 2 is that the blade 1321 in FIG. 2 has a plurality of cutting members 1322, which can increase the side-cutting capability of the multistep dental implant drill 1. The multistep dental implant drill 1 has a point angle θ, which is set at the top of the drill bit portion 13, and the angle of the point angle θ is 70 degrees to 120 degrees.

As shown in FIG. 3, a dental implant 2 comprises an implant fixture body 21 and a tooth root neck 22. The implant fixture body 21 has a fine thread portion 211, a coarse thread portion 212, and the plurality of side-cutting grooves 213; wherein the fine thread portion 211 located on the upper surface of the implant fixture body 21, and the coarse thread portion 212 located on the lower surface of the implant fixture body 21. Further, the fine thread portion 211 has a first screw pitch, the coarse thread portion 212 has a second screw pitch, and the second screw pitch is greater than the first screw pitch; wherein the length of the fine thread portion 211 is in a range of 5% to 15% based on the total length of the implant fixture body 21; and the length of the coarse thread portion 212 is in a range of 85% to 95% based on the total length of the implant fixture body 21. Moreover, the side-cutting grooves 213 are distributed on the coarse thread portion 212 and penetrate the outer surface of the implant fixture body 21; and the side-cutting grooves 213 extend from the bottom of the implant fixture body 21 to the bottom of the fine thread portion 211. The transverse width W1 of the side-cutting grooves 213 close to the bottom of the coarse thread portion 212 is greater than the transverse width W2 of the side-cutting grooves 213 close to the bottom of the fine thread portion 211. Furthermore, the tooth root neck 22 has a joint hole 221 and located on the top of the implant fixture body 21. Moreover, the difference between FIG. 3 and FIG. 4 is that the side-cutting grooves 213 of the dental implant 2 of FIG. 3 are rotationally distributed on the coarse thread portion 212, and the side-cutting grooves 213 of the dental implant 2 of FIG. 4 are vertically distributed on the coarse thread portion 212.

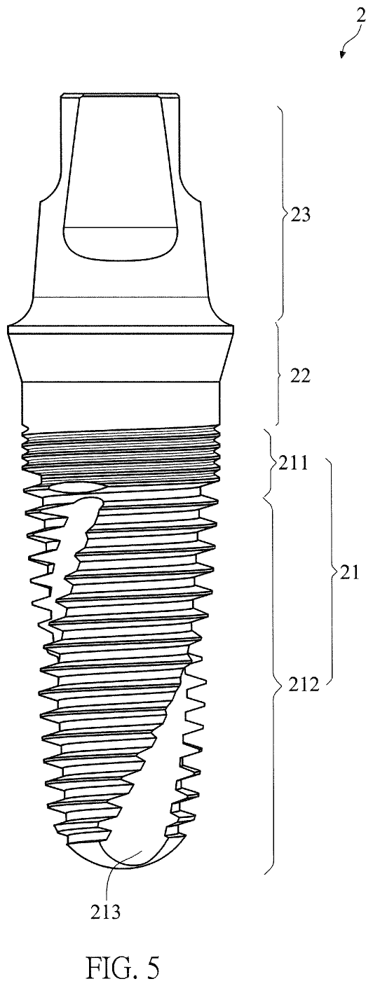

Please refer to FIG. 5, FIG. 5 is a schematic view of a dental implant according to Example 3 of the present invention.

As shown in FIG. 5, the dental implant 2 comprises the implant fixture body 21, the tooth root neck 22 and a second abutment 23. First, the implant fixture body 21 has a fine thread portion 211, a coarse thread portion 212 and at least one side-cutting groove 213. Further, the fine thread portion 211 is located on the upper surface of the implant fixture body 21; the coarse thread portion 212 portion is located on the lower surface of the implant fixture body 21; and the tooth root neck 22 is located on top of implant fixture body 21. Furthermore, the second abutment 23 is located on the top of the tooth root neck 22; the second abutment 23 is integrally formed with the implant fixture body 21; and the artificial crown is installed on the top of the second abutment 23. Furthermore, the at least one side-cutting groove 213 is distributed on the coarse thread portion 212 and penetrates the outer surface of the implant fixture body 21, and the side-cutting groove 213 extends from the bottom of the implant fixture body 21 to the bottom of the fine thread portion 211. Moreover, the length of the fine thread portion 211 is in a range of 5% to 15% based on the total length of the implant fixture body 21; and the length of the coarse thread portion 212 is in a range of 85% to 95% based on the total length of the implant fixture body 21.

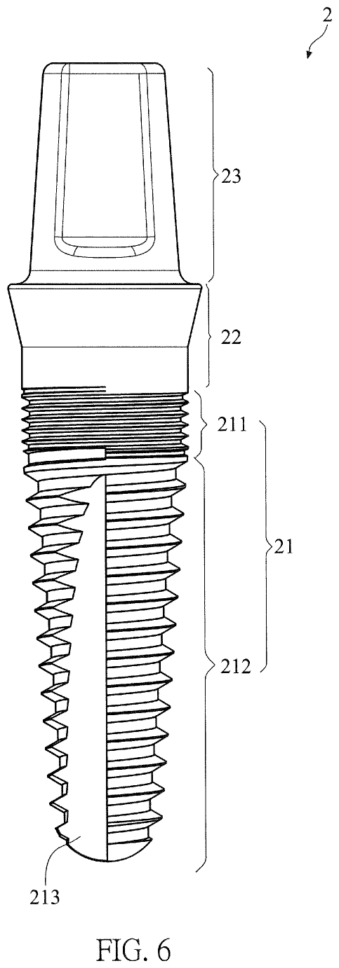

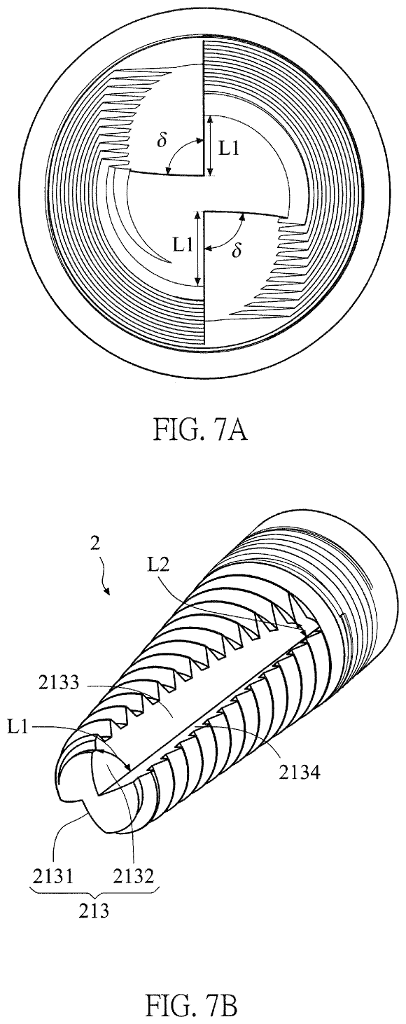

Please refer to FIG. 6, FIG. 6 is a schematic view of a dental implant according to Example 4 of the present invention.

As shown in FIG. 6, the embodiment is substantially the same as the dental implant 2 described in the previous embodiment. The difference is that the side-cutting groove 213 of the previous embodiment is rotationally distributed on the coarse thread portion 212 and penetrates outer surface of the implant fixture body 21. However, the side-cutting groove 213 of the embodiment is vertically distribute to the cause thread portion 212 and penetrates the outer surface of the implant fixture body 21.

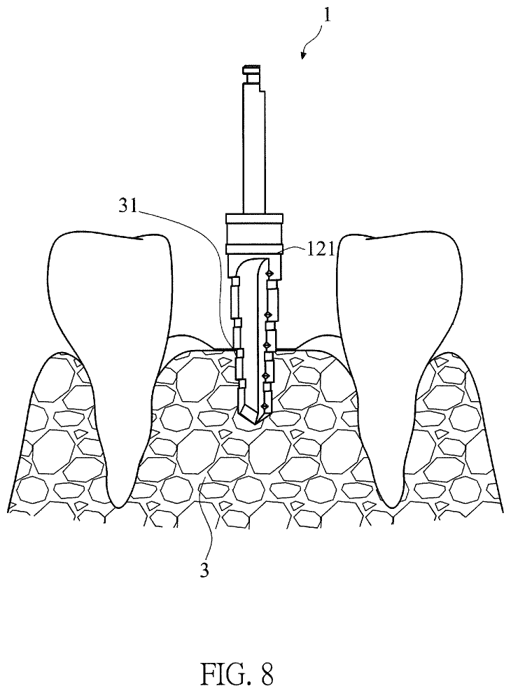

Please refer to FIG. 7A and FIG. 7B, FIG. 7A is a top view of the dental implant according to Example 1 of the present invention; and FIG. 7B is an another stereoscopical schematic view of the dental implant according to Example 1 of the present invention.

As shown in FIG. 7A and FIG. 7B, the implant fixture body 21 comprise the two side-cutting grooves 213, the two side-cutting grooves 213 are a first side-cutting groove 2131 and a second side-cutting groove 2132, respectively, and the first side-cutting groove 2131 is opposite to the second side-cutting groove 2132. The side-cutting grooves 213 has a cutting surface 2133 and a stacking surface 2134; wherein the cutting surface 2133 and the stacking surface 2134 penetrate vertically through the outer surface of the implant fixture body 21, and the cutting surface 2133 is disposed adjacent to the stacking surface 2134. In addition, the cutting surface 2133 and the stacking surface 2134 form an included angle δ, and the angle of the included angle δ is between 30 degrees and 90. In one aspect of the present invention, the implant fixture body 21 includes two side-cutting grooves 213; wherein the two side-cutting grooves 213 include the first side-cutting groove 2131 and the second side-cutting grooves 2132, and the included angle δ is 85 degrees to 90 degrees. In another aspect of present invention, the implant fixture body 21 includes three side-cutting grooves 213 which are the first side-cutting groove 2131, the second side-cutting groove 2132, and a third side-cutting groove (not shown in the figure). The included angle δ is between 55 degrees and 60, and the size of the included angle δ will affect the difficulty of lateral cutting of the coarse thread portion 212. As shown in FIG. 7A and FIG. 7B, the cutting surface 2133 and the stacking surface 2134 form a propeller shape for side-cutting capability, which makes the dental implant strongly lock, and when the dental implant is locked in the wrong position, the dental implant can be removed and locked again. Namely, counter clock turn the implant fixture body loose and redirect the angle relock it again to the preferred position. The first side-cutting groove 2131 and the second first side-cutting groove 2132 penetrate the outer surface of the implant fixture body 21, and the depth L1 of the first side-cutting groove 2131 and the second side-cutting groove 2132 at the bottom of the coarse thread portion 212 is greater than the depth L2 of the first side-cutting groove 2131 and the second side-cutting groove 2132 at the top of the coarse thread portion 212, so that the dental implant 2 has a tip at the bottom and has a better lateral cutting function. In addition, when the dental implant 1 is drilled and implanted in the alveolar bone, the first side-cutting groove 2131 and the second side-cutting groove 2132 will be filled with patient's own bone debris; and because the first side-cutting groove 2131 and the second side-cutting groove 2132 have deep grooves capable of filling with more patient's own bone debris to generate a relatively stable fixed structure for the dental implant 2.

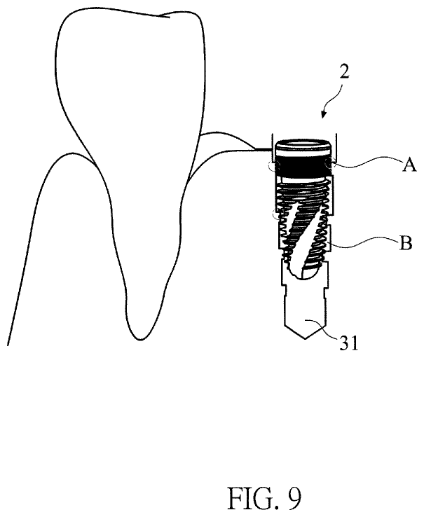

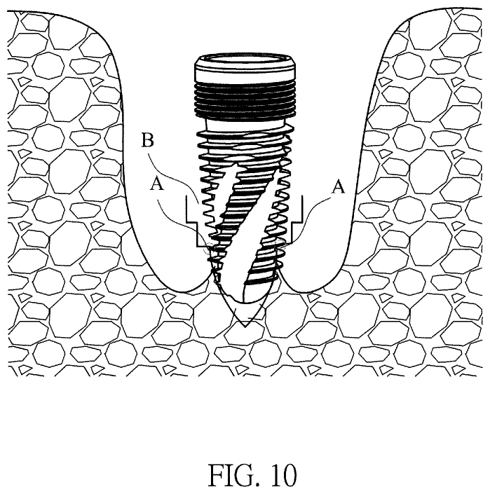

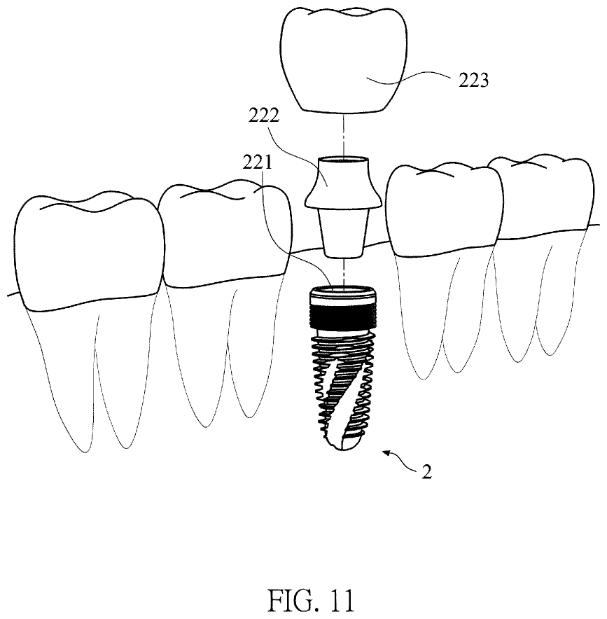

Please refer to FIG. 8 to FIG. 10, FIG. 8 is a schematic view of multistep dental implant drill drilling into alveolar bone according to the present invention; FIG. 9 is a schematic view of the dental implant implanted into an implant hole in the alveolar bone according to Example 1 of the present invention; FIG. 10 is a schematic view of the dental implant implanted into an immediate extracted tooth socket in the alveolar bone according to Example 1 of the present invention; and FIG. 11 is a schematic view of dental implant implanted into a healed alveolar bone according to the present invention.

As shown in FIG. 1, FIG. 2, FIG. 8, FIG. 10 and FIG. 11, a multistep dental implant drill 1 is aligned with an alveolar bone 3 at a preset implant position to drill an implant hole 31 and implant the dental implant 2; wherein the stopper 12 of the multistep dental implant drill 1 has a stopping line 121 and a stopping layer 122, and the dentist can judge the depth of the multistep dental implant drill 1 into the alveolar bone 3 through the stopping line 121. Further, the color of the stopping layer 122 is different from that of other parts and different color code for different width. When the multistep dental implant drill 1 is drilled into the alveolar bone 3 until the stopping line 121 contacts the alveolar bone 3 to complete drilling. Therefore, the stopping line 121 can avoid drilling too deeply to hurt the patient's facial nerve; alternatively, the dental implant cannot be implanted because of drilling too shallowly. Secondly, when the dental implant 2 is implanted in the implant hole 31, the dental implant 2 is tightly integrated with the implant hole to form a step locking concept (as shown in A of FIGS. 9 and 10); in contrast, the dental implant 2 and the implant hole cannot totally integrated (as shown in B of FIGS. 9 and 10). Therefore, the dental implant 2 is tightly integrated with the hole (as shown in A of FIGS. 9 and 10) to generate bone debris, and bone debris can be filled in empty places (as shown in B of FIGS. 9 and 10), so that the dental implant 2 has a good initial stability. Further, the joint hole 221 of the dental implant 2 is connected to a first abutment 222, and an artificial dental crown 223 is installed on the first abutment 222.

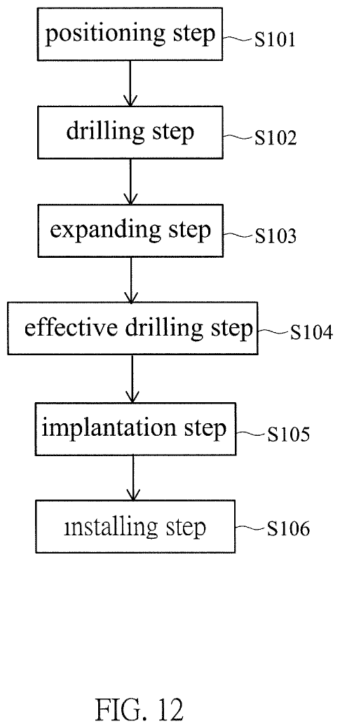

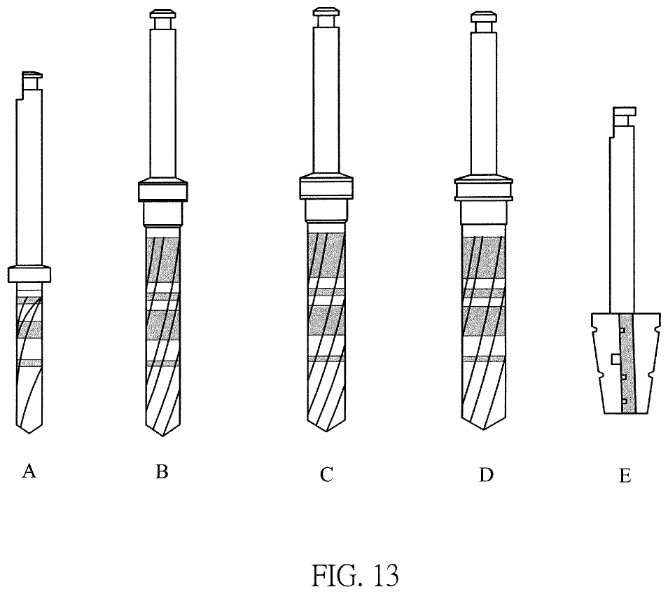

Please refer to FIG. 12, FIG. 12 is a flow chart of a method for using one single drill dental implant system of the present invention.

As shown in FIG. 12, a method for using one single drill dental implant system, comprising the steps of:

a positioning step S101 of aligning a tip of a drill bit portion of a multistep dental implant drill with a preset dental implant position;

a drilling step S102 of rotating the multistep dental implant drill by a dental handpiece to drill an implant hole in the alveolar bone;

an expanding step S103 of drilling the alveolar bone through a plurality of steps and a plurality of cutting members of the drill bit portion, so that the diameter of the implant hole is gradually enlarged to be smaller than the diameter of a dental implant;

a further drilling step S104 of using a stopping line of a stopper to determine the depth of the drill bit portion into the alveolar bone after the expanding step; and the drilling is completed when the stopping line contacts the alveolar bone;

an implantation step S105 of cutting the alveolar bone by a cutting surface of the side-cutting groove of the dental implant, and filling with bone debris in the side-cutting groove to fix the dental implant, so as to complete the implantation of the dental implant in the hole; and

an installing step S106 of connecting first abutment to a joint hole of the dental implant, and installing an artificial crown on the first abutment.

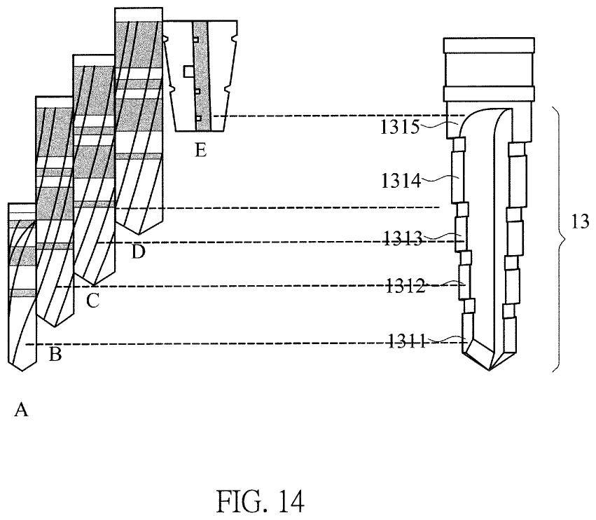

Please refer to FIG. 13 to FIG. 14, FIG. 13 is a schematic view of a set of dental implant drill required for drilling holes in current osteotomy; and FIG. 14 is a schematic view of multistep dental implant drill of the present invention formed by five different dental implant drills.

As shown in FIG. 13 to FIG. 14, at present, the osteotomy requires a variety of drill bits with different diameters, as shown in A to E in FIG. 13. Further, A, B, C and E are the drill bits with increasing diameters in sequence. After the drill bits are used sequentially, the implant hole with a diameter slightly smaller than the diameter of the dental implant can be drilled, for example, if the diameter of the dental implant is 4.85 mm, the diameter of the E drill bit is 4.35 mm. As shown in FIG. 14, A, B, C and E drill bits are combined to form the multistep dental implant drill of the present invention, which reduces the operation time, chances of infection, risk of excessive bleeding, patients discomfort, and post-operation complications.

The foregoing description, for the purpose of explanation, has been described with reference to specific embodiments; however. The embodiments were chosen and described in order to best explain the principles of the disclosure and its practical applications, to thereby enable others skilled in the art to best utilize the disclosure and various embodiments with various modifications as are suited to the particular use contemplated. The embodiments depicted above and the appended drawings are exemplary and are not intended to be exhaustive or to limit the scope of the disclosure to the precise forms disclosed. Modifications and variations are possible in view of the above teachings.

I claim:

1. One single drill dental implant system, comprising:
a multistep dental implant drill engaged with a dental handpiece and comprising a rod portion, a stopper and a drill bit portion, the stopper connected to the rod portion and the drill bit portion, the drill bit portion comprising a plurality of steps with a diameter decreasing from top to bottom, at least one cutting flute and at least one tip, and the cutting flute penetrating the outer surface of the drill bit portion and extending from the bottom of the drill bit portion to the bottom of the stopper; and
a dental implant comprising:
an implant fixture body having a fine thread portion, a coarse thread portion, and the plurality of side-cutting grooves; the fine thread portion located on the upper surface of the implant fixture body, the coarse thread portion located on the lower surface of the implant fixture body; the side-cutting grooves distributed on the coarse thread portion and penetrating the outer surface of the implant fixture body; and the side-cutting grooves extending from the bottom of the implant fixture body to the bottom of the fine thread portion;
a tooth root neck having a joint hole and located on the top of the implant fixture body;
wherein the tip of the multistep dental implant drill is aligned with a preset implant position on an alveolar bone to drill an implant hole and implant the dental implant;
wherein the length of the fine thread portion is in a range of 5% to 15% based on the total length of the implant fixture body; and the length of the coarse thread portion is in a range of 85% to 95% based on the total length of the implant fixture body.

2. The one single drill dental implant system mentioned in claim 1, wherein the multistep dental implant drill has a point angle which is set at the top of the drill bit portion, and the point angle is in a range of 70 degrees to 120 degrees.

3. The one single drill dental implant system mentioned in claim 1, wherein the multistep dental implant drill includes two cutting flutes to be respectively a first cutting flute and a second cutting flute, and the first cutting flute and the second cutting flute penetrate outer surface of the drill bit portion; the first and second cutting flutes extend from the bottom of the drill bit portion to the bottom of the stopper; and the second cutting flute is opposite to the first cutting flute.

4. The one single drill dental implant system mentioned in claim 3, wherein each of the plurality of steps and two sides of the first cutting flute and the second cutting flute form a plurality of blades.

5. The one single drill dental implant system mentioned in claim 4, wherein the plurality of blades have a plurality of cutting members.

6. The one single drill dental implant system mentioned in claim 1, wherein the stopper has a stopping line, and the stopping line is arranged around the bottom of the stopper.

7. A method for using one single drill dental implant system, comprising the steps of:
a positioning step of aligning a tip of a drill bit portion of a multistep dental implant drill with a preset implant position;
a drilling step of rotating the multistep dental implant drill by a dental handpiece to drill an implant hole in the alveolar bone;
an expanding step of drilling the alveolar bone through a plurality of steps and a plurality of cutting members of the drill bit portion, so that the diameter of the implant hole is gradually enlarged to be smaller than the diameter of a dental implant; and
an implantation step of implanting the dental implant into the implant hole.

8. The method mentioned in claim 7, further comprising a further drilling step of using a stopping line of a stopper to determine the depth of the drill bit portion into the alveolar bone after the expanding step; and the drilling is completed when the stopping line contacts the alveolar bone.

9. The method mentioned in claim 7, wherein the alveolar bone is cut by a cutting surface of the side-cutting grooves of the dental implant when the dental implant is implanted, and the side-cutting grooves is filled with bone debris and locks the dental implant.

10. The method mentioned in claim 7, wherein a first abutment is connected to a joint hole of the dental implant, and an artificial crown is installed on the first abutment after the implantation step.
